(19) United States
(12) Patent Application Publication
(10) Pub. No.: US 2006/0122694 A1
(43) Pub. Date: Jun. 8, 2006

(54) Medical devices and methods of making the same

(76) Inventors: Jonathan S. Stinson, Plymouth, MN (US); Daniel J. Gregorich, Mound, MN (US)

Correspondence Address: FISH & RICHARDSON PC, P.O. BOX 1022, MINNEAPOLIS, MN, 55440-1022, US

(21) Appl. No.: 11/004,009

(22) Filed: Dec. 3, 2004

(51) Int. Cl. A61F 2/06

(52) U.S. Cl. 623/1.34

(57) ABSTRACT

Medical devices, such as stents, and methods of making the devices, are described. In some embodiments, the invention features a medical device that includes a member having a first portion and a second portion that define an electrically conductive loop. The first portion is adapted to break or erode after expansion of the medical device, and the second portion is not adapted to break or erode after expansion of the medical device. The breaking or erosion of the first portion breaks the electrically conductive loop.

### TECHNICAL FIELD

The invention relates to medical devices, such as, for example, stents and stent-grafts, and methods of making the devices.

### BACKGROUND

The body includes various passageways such as arteries, other blood vessels, and other body lumens. These passageways sometimes become occluded or weakened. For example, the passageways can be occluded by a tumor, restricted by plaque, or weakened by an aneurysm. When this occurs, the passageways can be reopened or reinforced, or even replaced, with a medical endoprosthesis. An endoprosthesis is typically a tubular member that is placed in a lumen in the body. Examples of endoprostheses include stents, stent-grafts, and covered stents.

An endoprosthesis can be delivered inside the body by a catheter that supports the endoprosthesis in a compacted or reduced-size form as the endoprosthesis is transported to a desired site. Upon reaching the site, the endoprosthesis is expanded, for example, so that it can contact the walls of the lumen.

When the endoprosthesis is advanced through the body, its progress can be monitored (e.g., tracked), so that the endoprosthesis can be delivered properly to a target site. After the endoprosthesis has been delivered to the target site, the endoprosthesis can be monitored to determine whether it has been placed properly and/or is functioning properly.

Methods of tracking and monitoring a medical device include X-ray fluoroscopy and magnetic resonance imaging (MRI). MRI is a non-invasive technique that uses a magnetic field and radio waves to image the body. In some MRI procedures, the patient is exposed to a magnetic field, which interacts with certain atoms (e.g., hydrogen atoms) in the patient's body. Incident radio waves are then directed at the patient. The incident radio waves interact with atoms in the patient's body, and produce characteristic return radio waves. The return radio waves are detected by a scanner and processed by a computer to generate an image of the body.

### SUMMARY

In one aspect, the invention features a medical device, such as an implantable medical endoprosthesis (e.g., a stent), including a member having a first portion and a second portion that define an electrically conductive loop. The first portion is adapted to break or erode after expansion of the medical device, and the second portion is not adapted to break or erode after expansion of the medical device. The breaking or erosion of the first portion breaks the electrically conductive loop. In some embodiments, the medical device can have no electrically conductive loops after the first portion has broken or eroded. As explained below, this decrease in electrical continuity or lack of electrical continuity after the first portion has broken or eroded can enhance the visibility of material present in the lumen of the medical device during MRI. At the same time, before the first portion has eroded, the medical device can have a relatively high mechanical integrity (e.g., the medical device can be relatively strong), such that the medical device is capable of supporting a lumen of a subject. In another aspect, the invention features a medical device having a generally tubular member that includes at least one metal alloy selected from titanium-iridium (Ti—Ir), titanium-rhenium (Ti—Re), titanium-tantalum-iridium (Ti—Ta—Ir), and titanium-tantalum-rhenium (Ti—Ta—Re).

In an additional aspect, the invention features a method that includes expanding a medical device including a first portion and a second portion that define an electrically conductive loop. After the medical device has been expanded, the first portion breaks or erodes and thereby breaks the electrically conductive loop.

In a further aspect, the invention features a method that includes delivering a medical device into a lumen of a subject. The medical device includes a member having a first portion and a second portion that define an electrically conductive loop. The first portion is adapted to break or erode after expansion of the medical device, and the second portion is not adapted to break or erode after expansion of the medical device.

In another aspect, the invention features a method that includes expanding a medical device having at least one electrically conductive loop to break the at least one electrically conductive loop.

Embodiments may include one or more of the following features.

The medical device can be radiopaque. In certain embodiments, the medical device can include an alloy that includes one or more of the following metals: titanium, vanadium, tantalum, zirconium, niobium, molybdenum, platinum, palladium, aluminum, iridium, rhenium, and tungsten. For example, the medical device can include titanium-molybdenum, titanium-niobium-tantalum-zirconium, titanium-tantalum, titanium-aluminum-vanadium-tantalum, titanium-iridium, titanium-rhenium, titanium-tantalum-iridium, titanium-tantalum-rhenium, and/or niobium-zirconium.

The medical device (e.g., the first portion) can include a bioerodible material, such as a metal. In some embodiments, the medical device can include magnesium, titanium, zirconium, niobium, tantalum, zinc, silicon, lithium, sodium, potassium, manganese, calcium, iron, or a combination thereof.

The medical device can include a material (e.g., a metal, a metal alloy) having a magnetic susceptibility of less than about 0.9×10⁻³, and/or a density of greater than about eight grams per cubic centimeter (e.g., greater than about 9.9 grams per cubic centimeter).

The medical device (e.g., the first portion) can further include an oxide.

The thickness of the second portion can be greater than the thickness of the first portion.

The medical device can be an implantable medical endoprosthesis (e.g., a stent). The implantable medical endoprosthesis can include at least one band or strut defining a hole, a notch, a slot, a groove, or a chamfer. Alternatively or additionally, the implantable medical endoprosthesis can include at least one band or strut having a first region with a first thickness and a second region with a second thickness that is greater than the first thickness.

The method can further include expanding the medical device. After the medical device has been expanded, the first portion can break or erode and thereby break the electrically conductive loop. In some embodiments, the electrically conductive loop can be broken from about one week to about three weeks after the medical device has been expanded. In certain embodiments, the electrically conductive loop can be broken from about one month to about three months after the medical device has been expanded. In some embodiments, the electrically conductive loop can be broken from about six months to about nine months after the medical device has been expanded. After the first portion has broken or eroded, the medical device may not define any electrically conductive loops. The method can further include, after the medical device has been expanded, expanding the medical device again so that the electrically conductive loop breaks. The medical device can be expanded using a medical balloon. In certain embodiments, the medical device can be exposed to ultrasound after the medical device has been expanded.

The method can further include altering a configuration of the medical device so that the electrically conductive loop breaks. Altering a configuration of the medical device can include breaking at least one component (e.g., a band, a strut) of the medical device. Altering a configuration of the medical device can include heating and/or cooling a portion of the medical device. In some embodiments, altering a configuration of the medical device can include contacting a portion of the medical device with an agent that dissolves the portion of the medical device.

The method can further include viewing the medical device and/or the lumen of the subject with magnetic resonance imaging. Alternatively or additionally, the method can further include viewing the medical device using X-ray fluoroscopy.

Embodiments may have one or more of the following advantages.

The medical device can allow material that is present within its lumen to be viewed using MRI, a non-invasive procedure, after the medical device has been delivered to a target site. Thus, an operator (e.g., a physician) can assess the condition of the target site (e.g., for signs of restenosis) after implantation of the medical device (e.g., two weeks after implantation, one month after implantation). In embodiments in which the medical device is radiopaque, the medical device can also be viewed using X-ray fluoroscopy (e.g., during delivery to a target site). In some embodiments, the medical device can have a relatively low profile prior to expansion, which can enhance the deliverability of the medical device (e.g., by making it easier to maneuver the medical device through a tortuous and/or narrow lumen). The medical device can have an electrically continuous strut and band pattern during manufacture, which can allow the medical device to be manufactured relatively efficiently and/or inexpensively, and can also prevent the medical device from experiencing substantial geometric distortion during loading onto a delivery device and during delivery to a target site. The medical device can have a generally tubular shape during initial use at a target site (e.g., prior to the formation of discontinuities in the strut and band geometry of the medical device), which can enhance the ability of the medical device to limit restenosis and/or provide uniform support to the target site. In certain embodiments, one or more electrical discontinuities can be formed in the medical device without adversely affecting the target site. For example, one or more electrical discontinuities can be formed in the medical device via the erosion and/or absorption of bioerodible segments in the medical device.

Other aspects, features and advantages of the invention will be apparent from the description of the preferred embodiments and from the claims.

### DESCRIPTION OF DRAWINGS

FIG. 1A is a perspective view of an embodiment of a stent.

FIG. 1B is a side cross-sectional view of section 1B of the stent of FIG. 1A.

FIG. 1C is a perspective view of the stent of FIG. 1A.

FIGS. 2A and 2B are illustrations of the stent of FIG. 1A within a lumen of a subject.

FIG. 3A is a side cross-sectional view of an embodiment of a stent band.

FIG. 3B is a side cross-sectional view of an embodiment of a stent band.

FIG. 3C is a side cross-sectional view of an embodiment of a stent band.

FIG. 3D is a perspective view of an embodiment of a stent band.

FIG. 3E is a side cross-sectional view of an embodiment of a stent band.

FIG. 3F is a perspective view of an embodiment of a stent band.

FIG. 3G is a cross-sectional view of the stent band of FIG. 3F, taken along line 3G-3G.

### DETAILED DESCRIPTION

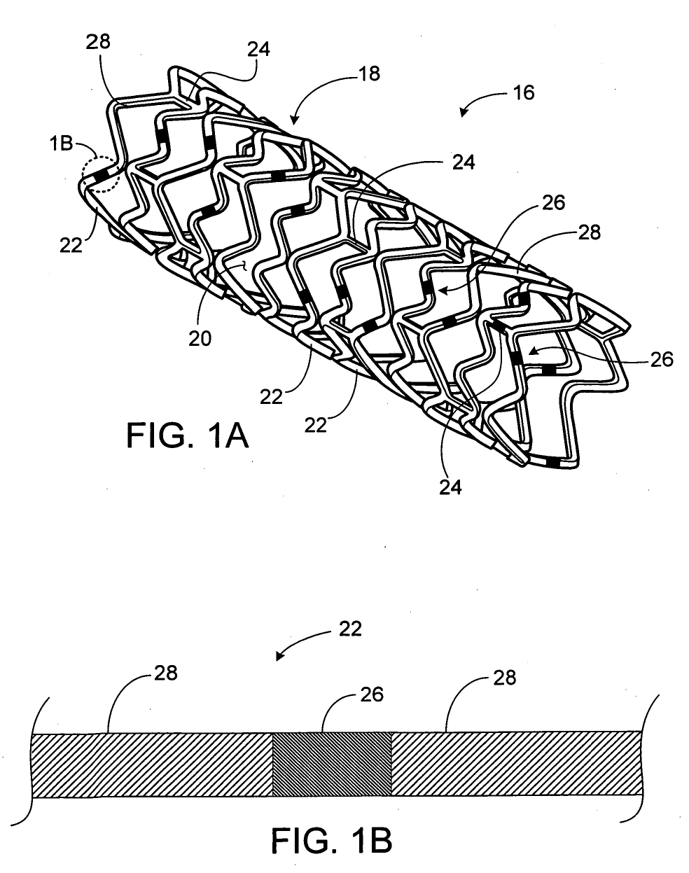

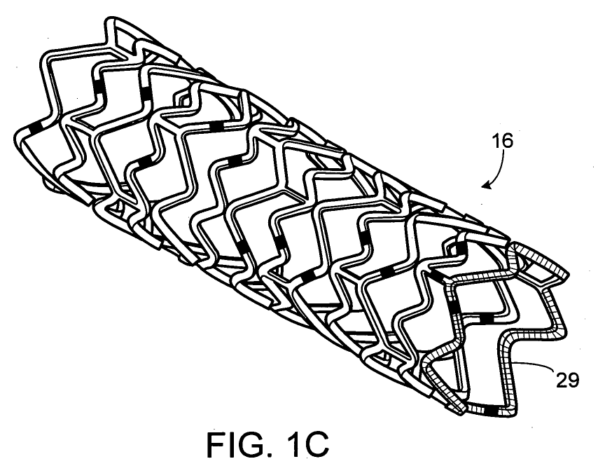

Referring to FIGS. 1A and 1B, a stent 16 includes a generally tubular body 18 defining a lumen 20. Generally tubular body 18 is formed of bands 22 that are connected by struts 24. As shown, both bands 22 and struts 24 include electrically conductive bioerodible portions 26 and non-bioerodible portions 28, which enhance the mechanical characteristics of stent 16. Since portions 26 and portions 28 are electrically conductive, they form electrically conductive loops, such as the electrically conductive loop 29 shown in FIG. 1C. These electrically conductive loops can adversely affect the MRI compatibility of stent 16. However, by removing bioerodible portions 26 at a selected time after stent 16 has been implanted, stent 16 is capable of providing both mechanical performance and MRI compatibility.

As noted above, the presence of electrically conductive loops in a stent can adversely affect the MRI-compatibility of the stent. Without wishing to be bound by theory, it is believed that when a stent with electrically conductive loops is exposed to MRI, the electrically conductive loops can conduct a current that limits the visibility of material within the lumen of the stent. Specifically, during MRI, an incident electromagnetic field is applied to a stent. The magnetic environment of the stent can be constant or variable, such as when the stent moves within the magnetic field (e.g., from a beating heart) or when the incident magnetic field is varied. When there is a change in the magnetic environment of the stent, which can act as a coil or a solenoid, an induced electromotive force (emf) is generated, according to Faraday's Law. The induced emf in turn can produce an eddy current that induces a magnetic field that opposes the change in magnetic field. The induced magnetic field can interact with the incident magnetic field to reduce (e.g., distort) the visibility of material in the lumen of the stent. A similar effect can be caused by a radiofrequency pulse applied during MRI. Thus, the ability to use MRI to view and assess the condition of a target site that includes a stent such as stent 16 can be limited.

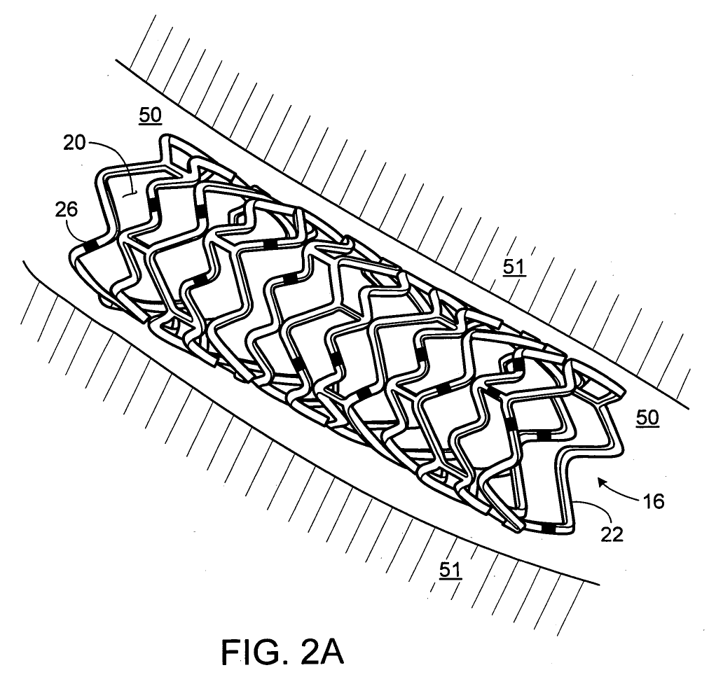

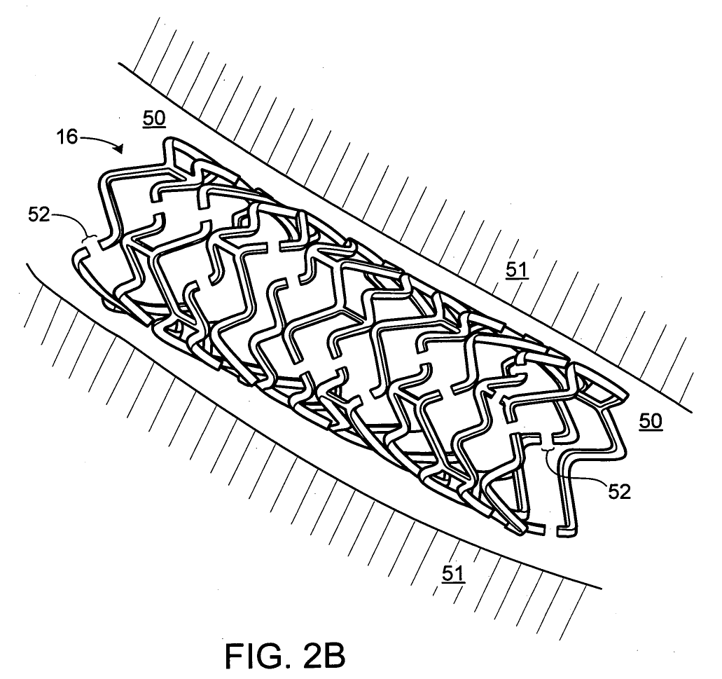

FIG. 2A shows stent 16 when the stent is disposed within a lumen 50 (e.g., an artery) of a subject. Stent 16 can be delivered to lumen 50 and expanded within lumen 50 using, for example, a stent delivery system such as a balloon catheter system. Catheter systems are described in, for example, Wang, U.S. Pat. No. 5,195,969, and Hamlin, U.S. Pat. No. 5,270,086. Stents and stent delivery are also exemplified by the Radius® or Symbiot® systems, available from Boston Scientific Scimed, Maple Grove, Minn. The shape and structure of stent 16 can allow the stent to be delivered into lumen 50 relatively easily. Stent 16 has a somewhat symmetrical tubular shape, allowing it to be loaded onto a delivery device relatively easily, and a relatively low profile, allowing it to be navigated through lumen 50 relatively easily.

As described above, the electrically conductive loops in stent 16 limit the MRI visibility of material within lumen 20 of stent 16. However, the generally tubular and somewhat symmetrical structure of stent 16 provides good support for lumen 50, and uniformly transfers stress away from lumen 50. Furthermore, over time, tissue from wall 51 of lumen 50 can grow over stent 16, effectively anchoring stent 16 into lumen 50.

Referring now to FIG. 2B, as times passes, the body erodes and/or absorbs bioerodible portions 26 of stent 16. Eventually, this erosion and/or absorption causes electrical discontinuities 52 to form in bands 22. In some embodiments, breathing can place a repeated stress on the stent that can contribute to the formation of electrical discontinuities 52 by causing bioerodible portions 26 to break. Electrical discontinuities 52 break the electrically conductive loops in stent 16 by disrupting the flow of electrical current through bands 22. As the number of electrically conductive loops in stent 16 decreases, the occurrence of an eddy current in stent 16 is reduced (e.g., eliminated). Accordingly, the occurrence of an induced magnetic field that can interact with the incident magnetic field is also reduced. As a result, the MRI visibility of material in the lumen of stent 16 can increase. At the same time, the anchoring of stent 16 into lumen 50 by tissue from wall 51 limits the likelihood that stent 16 will collapse or significantly distort as the electrical discontinuities form. As a result, stent 16 continues to provide sufficient support for lumen 50. In some embodiments, the formation of electrical discontinuities 52 in stent 16 may decrease the extent to which stent 16 shields lumen 50 from stress. However, this decrease in stress shielding can benefit lumen 50 by encouraging the lumen's tissue to remodel and strengthen.

As described above, stent 16 includes both bioerodible portions and non-bioerodible portions. The non-bioerodible portions of stent 16 can be formed of any MRI-compatible biocompatible material, such as a non-ferromagnetic material. As an example, the non-bioerodible portions of stent 16 can be formed of one or more materials with a relatively low magnetic susceptibility. For example, the non-bioerodible portions of stent 16 can be formed of a material (e.g., a metal or a metal alloy) with a magnetic susceptibility of less than 0.9×10⁻³ (e.g., less than 0.871×10⁻³, less than 0.3×10⁻³, less than −0.2×10⁻³). In certain embodiments, stent 16 can include biocompatible material with a magnetic susceptibility that is lower than the magnetic susceptibility of stainless steel and/or Nitinol. In some embodiments, a material with a relatively low magnetic susceptibility can be unlikely to move substantially and/or to experience a significant increase in temperature (e.g., a temperature increase of at least about 1° C.) as a result of being exposed to MRI.

The non-bioerodible portions of stent 16 can include a biocompatible material that can be used in a self-expandable stent, a balloon-expandable stent, or both. In embodiments in which stent 16 is a self-expandable stent, stent 16 can include a relatively elastic biocompatible material, such as a superelastic or pseudo-elastic metal alloy. Such materials can cause stent 16 to be relatively flexible during delivery, thereby allowing stent 16 to be safely advanced through a lumen (e.g., through a relatively tortuous vessel). Alternatively or additionally, such materials can allow stent 16 to temporarily deform (e.g., upon experiencing a temporary extrinsic load), and then regain its shape (e.g., after the load has been removed), without experiencing a permanent deformation, which could lead to re-occlusion, embolization, and/or perforation of the lumen wall. Examples of superelastic materials include a Nitinol (e.g., 55% nickel, 45% titanium), silver-cadmium (Ag—Cd), gold-cadmium (Au—Cd), gold-copper-zinc (Au—Cu—Zn), copper-aluminum-nickel (Cu—Al—Ni), copper-gold-zinc (Cu—Au—Zn), copper-zinc (Cu—Zn), copper-zinc-aluminum (Cu—Zn—Al), copper-zinc-tin (Cu—Zn—Sn), copper-zinc-xenon (Cu—Zn—Xe), indium-thallium (In—Tl), nickel-titanium-vanadium (Ni—Ti—V), titanium-molybdenum (Ti—Mo), titanium-niobium-tantalum-zirconium (Ti—Nb—Ta—Zr), and copper-tin (Cu—Sn). See, e.g., Schetsky, L. McDonald, “Shape Memory Alloys”, Encyclopedia of Chemical Technology (3rd ed.), John Wiley & Sons, 1982, vol. 20. pp. 726-736, for a full discussion of superelastic alloys. Other examples of materials include one or more precursors of superelastic alloys, i.e., those alloys that have the same chemical constituents as superelastic alloys, but have not been processed to impart the superelastic property under the conditions of use. Such alloys are further described in PCT Application No. US91/02420.

In certain embodiments, stent 16 can include one or more materials that can be used for a balloon-expandable stent, such as noble metals (e.g., platinum, gold, palladium), refractory metals (e.g., tantalum, tungsten, molybdenum, rhenium), and alloys thereof. Other examples of stent materials include titanium, titanium alloys (e.g., alloys containing noble and/or refractory metals), vanadium alloys, stainless steels, stainless steels alloyed with noble and/or refractory metals, nickel-based alloys (e.g., those that contain platinum, gold, and/or tantalum), iron-based alloys (e.g., those that contain platinum, gold, and/or tantalum), cobalt-based alloys (e.g., those that contain platinum, gold, and/or tantalum), aluminum alloys, zirconium alloys, and niobium alloys. For example, stent 16 can include titanium-tantalum (Ti—Ta), titanium-aluminum-vanadium-tantalum (Ti—Al—V—Ta), titanium-iridium (Ti—Ir), titanium-rhenium (Ti—Re), titanium-tantalum-iridium (Ti—Ta—Ir), titanium-tantalum-rhenium (Ti—Ta—Re), and/or niobium-zirconium (Nb—Zr). Metal alloys are described, for example, in U.S. Ser. No. 10/672,891, filed on Sep. 26, 2003, and entitled “Medical Devices and Methods of Making Same”.

In some embodiments, stent 16 can include one or more radiopaque materials (e.g., metals, metal alloys), which can cause stent 16 to be visible using X-ray fluoroscopy (e.g., allowing stent 16 to be tracked as it is delivered to a target site). Examples of radiopaque materials include metallic elements having atomic numbers greater than 26 (e.g., greater than 43), and/or those materials having a density greater than about eight grams per cubic centimeter (e.g., greater than about 9.9 grams per cubic centimeter, at least about 25 grams per cubic centimeter, at least about 50 grams per cubic centimeter). In some embodiments, a medical device can include a material (e.g., a metal, a metal alloy) with a magnetic susceptibility of less than 0.9×10⁻³ and a density of greater than about eight grams per cubic centimeter. For example, a medical device can include platinum, tantalum, palladium, and/or molybdenum. In certain embodiments, a radiopaque material can be relatively absorptive of X-rays. For example, the radiopaque material can have a linear attenuation coefficient of at least 25 cm⁻¹ (e.g., at least 50 cm⁻¹) at 100 keV. Examples of radiopaque materials include tantalum, platinum, iridium, palladium, tungsten, gold, ruthenium, niobium, and rhenium. The radiopaque material can include an alloy, such as a binary, a ternary or more complex alloy, containing one or more elements listed above with one or more other elements such as iron, nickel, cobalt, or titanium. The radiopaque material can, for example, be more radiopaque than stainless steel. In some embodiments, the radiopaque material can be more radiopaque than iron and/or Nitinol.

The bioerodible portions of stent 16 (e.g., portions 26) can be formed of one or more bioerodible materials, such as bioerodible metals and bioerodible metal alloys. Examples of bioerodible metal alloys include metal alloys that have at least one metal selected from the group of alkali metals, alkaline earth metals, iron, zinc, or aluminum. In some embodiments, a bioerodible metal alloy can include at least one metal selected from magnesium, titanium, zirconium, niobium, tantalum, zinc, and silicon, and/or at least one metal selected from lithium, sodium, potassium, manganese, calcium, and iron. For example, a bioerodible metal alloy can be a lithium-magnesium alloy, a sodium-magnesium alloy, or a zinc-calcium alloy. Other examples of bioerodible metal alloys include zinc-titanium alloys (e.g., zinc-titanium alloys including from about 0.1 percent by weight to about one percent by weight titanium, zinc-titanium-gold alloys including from about 0.1 percent by weight to about two percent by weight gold). In some embodiments, a bioerodible metal alloy can include cobalt, nickel, chromium, copper, cadmium, lead, tin, thorium, silver, gold, palladium, platinum, rhenium, carbon, and/or sulfur. Bioerodible materials are described, for example, in Bolz et al., U.S. Pat. No. 6,287,332, and U.S. Patent Application Publication No. US 2002/0004060 A1, published on Jan. 10, 2002.

In certain embodiments, bioerodible portions 26 can include a metal or a metal alloy capable of interacting with the material of non-bioerodible portions 28 such that the metal or metal alloy of bioerodible portions 26 selectively corrodes. For example, the material of bioerodible portions 26 can have a higher oxidation potential than the material of non-bioerodible portions 28, such that upon exposure to the electrolytic environment of the body, bioerodible portions 26 can galvanically corrode. Examples of combinations of materials include iron and copper; tantalum and iron; platinum and iron; tantalum and magnesium; platinum and magnesium; tantalum and aluminum; platinum and aluminum; and copper and stainless steel.

The erosion and/or absorption of bioerodible portions 26 of stent 16, and the corresponding formation of electrical discontinuities 52, can occur over a length of time that allows stent 16 to be delivered to a target site and expanded before a significant number of electrical discontinuities have been formed (e.g., before any electrical discontinuities have been formed). In some embodiments, one or more bioerodible portions 26 of stent 16 can be eroded and/or absorbed over a period of at least about one week (e.g., at least about two weeks, at least about three weeks, at least about one month, at least about two months, at least about three months, at least about four months, at least about five months, at least about six months, at least about seven months, at least about eight months), and/or at most about nine months (e.g., at most about eight months, at most about seven months, at most about six months, at most about five months, at most about four months, at most about three months, at most about two months, at most about one month, at most about three weeks, at most about two weeks).

Stent 16 can be of any desired shape and size (e.g., a coronary stent, an aortic stent, a peripheral vascular stent, a gastrointestinal stent, a urology stent, a neurology stent). Depending on the application, stent 16 can have an expanded diameter of, for example, from about one millimeter to about 46 millimeters. In certain embodiments, a coronary stent can have an expanded diameter of from about 1.5 millimeters to about six millimeters (e.g., from about two millimeters to about six millimeters). In some embodiments, a peripheral stent can have an expanded diameter of from about four millimeters to about 24 millimeters. In certain embodiments, a gastrointestinal and/or urology stent can have an expanded diameter of from about six millimeters to about 30 millimeters. In some embodiments, a neurology stent can have an expanded diameter of from about one millimeter to about 12 millimeters. An abdominal aortic aneurysm (AAA) stent and a thoracic aortic aneurysm (TAA) stent can have an expanded diameter from about 20 millimeters to about 46 millimeters. Stent 16 can be balloon-expandable, self-expandable, or a combination of both (e.g., Andersen et al., U.S. Pat. No. 5,366,504).

While a stent with bioerodible portions has been described, in some embodiments, as an alternative to or in addition to bioerodible portions, a stent can include one or more other types of weak regions. The weak regions can include, for example, one or more notches, slots, holes, thinned areas, grooves, and/or chamfers. The weak regions can be formed, for example, in a band and/or strut of the stent. Over time, strain on these weak regions (e.g., as a result of vessel pressure pulsation or peristalsis) can result in metal fatigue, which can eventually cause the weak regions to break apart. Alternatively or additionally, the weak regions can be mechanically broken apart. For example, after a stent has been implanted at a target site and a desired amount of time (e.g., six months) has passed, a balloon can be inserted into the stent (e.g., using a balloon catheter) and expanded until the weak regions break.

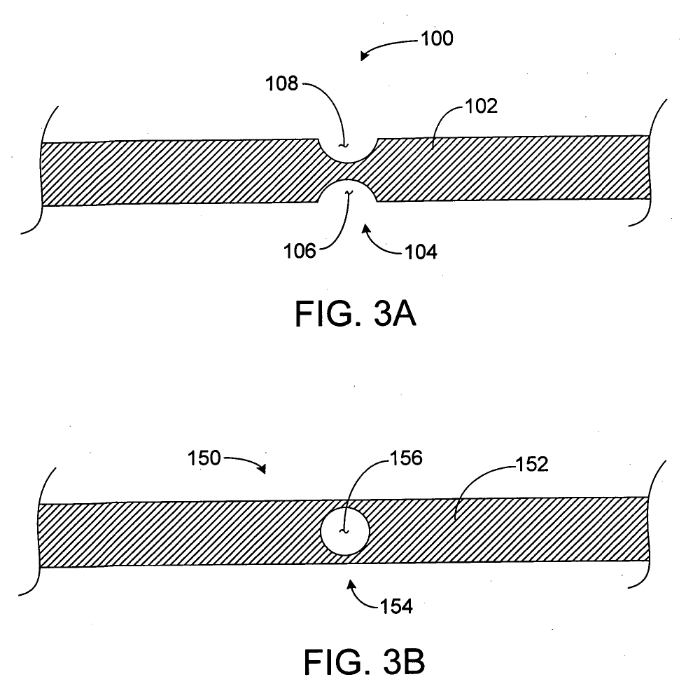

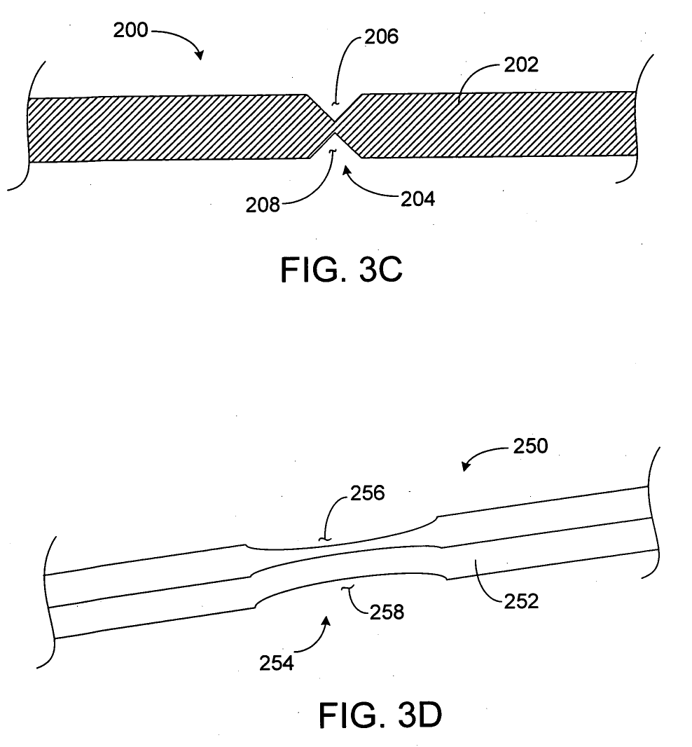

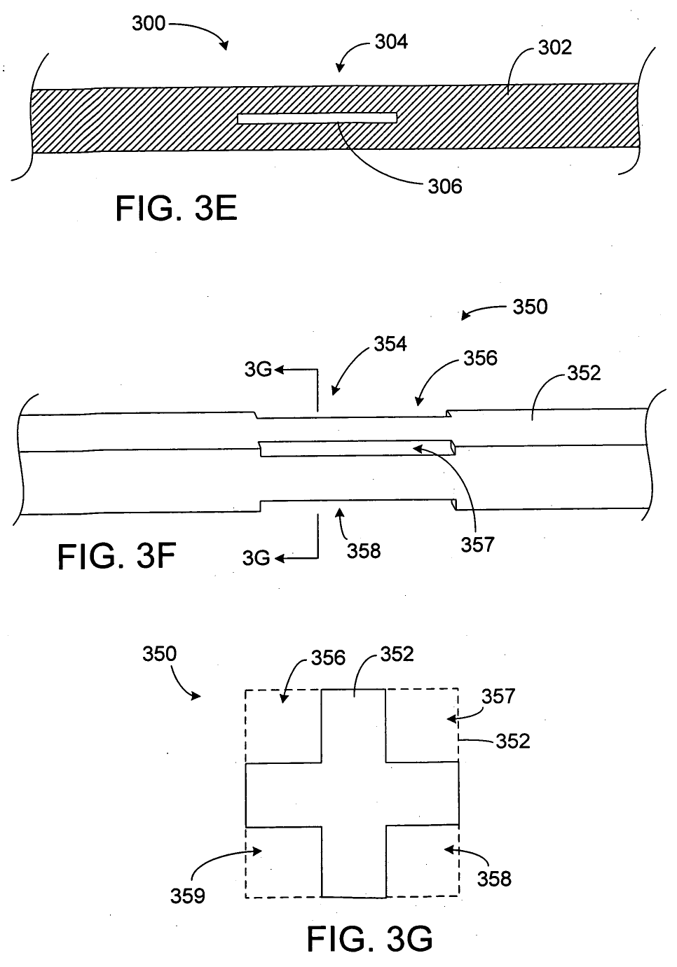

As an example, FIG. 3A shows a portion of a stent 100 that includes a band 102 with a weak region 104 formed of two hemispherical notches 106 and 108. After stent 100 has been delivered to a target site, it can be expanded. This expansion can bend and/or stretch the stent, further weakening weak region 104. After a certain amount of time, weak region 104 can break, thereby forming an electrical discontinuity in band 102. In certain embodiments, weak region 104 may be further weakened or broken by other methods, such as exposure of weak region 104 to ultrasound, or additional expansion of stent 100 (e.g., by a balloon). In some embodiments, the environment of the target site, such as the pressure of blood flow through the target site, can cause weak region 104 to break.

Other types of weak regions can be used. As an example, FIG. 3B shows a portion of a stent 150 that has a band 152 with a weak region 154 including a hole 156. In certain embodiments, hole 156 can be filled with a bioerodible material that can erode upon delivery of stent 150 to a target site. The bioerodible material can, for example, temporarily enhance the strength of weak region 154 during delivery to the target site. As another example, FIG. 3C shows a portion of a stent 200 including a band 202 with a weak region 204 formed out of two V-shaped notches 206 and 208. As an additional example, FIG. 3D shows a portion of a stent 250 that includes a band 252 with a weak region 254 formed out of two grooves 256 and 258. As a further example, FIG. 3E shows a portion of a stent 300 that includes a band 302 with a weak region 304 including a slot (a relatively long narrow opening) 306. As another example, FIGS. 3F and 3G show a portion of a stent 350 that includes a band 352 with a weak region 354 formed out of chamfers 356, 357, 358, and 359.

In some embodiments, a weak region of a stent can include an oxide (e.g., a metal oxide). For example, a weak region of a stent can include an oxide layer. In certain embodiments, an oxide layer can be formed in a region of a stent by selectively enriching the region of the stent with oxygen. For example, a localized region of a metal stent (e.g., a section of a strut) can be isolated by covering other regions of the stent with a protective layer. The metal stent can include, for example, tantalum, niobium, titanium, and/or molybdenum. After a localized region of the stent has been isolated, the stent can be heated (e.g., at a temperature of from about 300° C. to about 800° C.) in an atmosphere that includes oxygen, such that the localized region of the stent is oxidized. Thereafter, the protective layer can be removed from the stent (e.g., by dissolution). Other methods can be used to form an oxide layer on a stent. As an example, a metal stent can be heated (e.g., at a temperature of from about 300° C. to about 800° C.) in an atmosphere that includes oxygen, such that the entire stent surface is oxidized. Thereafter, certain regions of the stent can be covered with a protective layer, and the oxide layer on the stent can be removed from the regions of the stent that are not covered by the protective layer. The oxide layer can be removed, for example, by dissolution (e.g., electropolishing) and/or chemical etching (e.g., chemical milling). After the oxide layer has been removed from the unprotected regions of the stent, the protective layer can be removed from the stent, revealing the regions of the stent that still have an oxide layer. As another example, an oxide layer can be formed on a selected region of a stent by treating that region of the stent with a laser in the presence of a fluid or a gas that includes oxygen. In certain embodiments, an oxide layer can be formed by an anodization process. Anodization is described, for example, in U.S. Ser. No. 10/664,679, filed on Sep. 16, 2003, and entitled “Medical Devices”.

In certain embodiments, one or more regions of a stent can be weakened and/or embrittled by selectively exposing the regions to hydrogen and/or nitrogen (e.g., using one or more of the methods described above with reference to forming an oxide layer on a selected region of a stent). Exposure of a region of a stent to hydrogen can result in the formation of a hydride that weakens the region, and exposure of a region of a stent to nitrogen can result in the formation of a nitride that weakens the region.

In some embodiments, a weak region in a metal stent can include carbide particles. The carbide particles can be added to the stent by, for example, melting the stent material and adding solid carbon to the stent material in its melted state, and/or heat-treating the stent material in a gaseous atmosphere containing carbon (e.g., CO, CO₂). In certain embodiments, a weak region in a stent can include a carbide layer. A carbide layer can be formed on a metal stent by, for example, heat-treating the stent material in a gaseous atmosphere containing carbon. Alternatively or additionally, a carbide layer can be formed on a metal stent by a pack diffusion process, in which the stent material is contacted with a carbon-containing solid material, so that carbon diffuses in the solid state from the carbon-containing solid material into the stent material. Pack diffusion is described, for example, in ASM Handbook Vol. 4: Heat Treating (ASM International, 1991), pages 325-328.

In certain embodiments during the formation of an oxide, hydride, nitride, or carbide weak region, any of the above-described processes can be monitored to ensure that the oxide, hydride, nitride, or carbide does not extend too deeply into the stent material. In some embodiments, tests can be performed to determine the desirable depth to which an oxide, hydride, nitride, or carbide layer should be formed. For example, different thicknesses of oxide, hydride, nitride, and/or carbide layers can be formed on coupons of a metal stent material that have the same thickness as a metal stent strut. Testing can then be performed on the coupons to measure the yield strength and elongation at fracture. The results of the testing can be evaluated in light of the extent of strain a stent strut is expected to experience while in service. The coupon that produces a fracture within that strain value can be selected. For example, if the stent strain limit during expansion is 30 percent, an oxide, hydride, nitride, or carbide layer depth that produces localized fracture at 20 percent strain can be selected. In some embodiments, there can be a difference between the strut strain upon expansion and the strut strain when the fracture occurs, such that the fracture faces move apart from each other as the stent continues to strain beyond the value at which the fracture occurs. Separation between the fracture faces can, for example, provide a gap that is sufficient to avoid electrical conductivity between the two fracture faces.

In some embodiments, a weak region can be formed on a metal stent (e.g., a stent including tantalum, niobium, titanium, molybdenum) by forming a brittle intermetallic phase on the stent. The intermetallic phase can include, for example, niobium and rhenium (e.g., from 45 percent by weight to 65 percent by weight rhenium), niobium and rhodium (e.g., from 28 percent by weight to 38 percent by weight rhodium), niobium and silicon, or titanium and zinc (e.g., more than about five percent by weight zinc). In certain embodiments, an intermetallic phase can be formed by applying a second metal (e.g., rhenium, rhodium, silicon) to a localized area of a metal (e.g., niobium) stent in solid form, and then heating the second metal to allow it to diffuse into the metal stent and form the brittle phase. The second metal can be applied to the stent by, for example, adhering metal powder to the localized region (e.g., a strut) of the stent.

The above-described stents can be formed by any of a number of different methods. In some embodiments, a weak region (e.g., a notch, a hole) can be formed in a stent by laser cutting (e.g., using an excimer laser and/or an ultrashort pulse laser). Laser cutting is described, for example, in Saunders, U.S. Pat. No. 5,780,807, and Weber, U.S. Pat. No. 6,517,888. Other methods of forming a weak region include mechanical machining (e.g., micro-machining), electrical discharge machining (EDM), photoetching (e.g., acid photoetching), and/or chemical etching. In certain embodiments, a weak region can be formed in a stent by bending and/or twisting the material (e.g., metal) used to form the stent prior to forming the stent. In embodiments in which a stent includes one or more bioerodible portions, the bioerodible portions can be formed by cutting the stent using one of the above-described methods to form a discontinuity, and filling the discontinuity with a bioerodible material. The bioerodible material can be bonded to the stent by, for example, an adhesive (e.g., acrylic, cyanoacrylate, epoxy, polyurethane). Alternatively or additionally, the bioerodible material can be bonded to the stent using ultrasonic welding, laser welding, ultraviolet bonding, and/or heat bonding. In certain embodiments, the bioerodible material can be bonded to the stent by suspending the bioerodible material in a substrate (e.g., styrene-isobutylene-styrene) that is attached to and/or coated on the stent.

In some embodiments, a stent can further be finished (e.g., electropolished) to a smooth finish, according to conventional methods. In certain embodiments, at least about 0.0001 inch (e.g., about 0.0005 inch) of material can be removed from the interior and/or exterior surfaces of a stent by chemical milling and/or electropolishing. In some embodiments, a stent can be annealed at predetermined stages to refine the mechanical and physical properties of the stent.

While certain embodiments have been described, other embodiments are possible.

As an example, in some embodiments, an electrical discontinuity can be formed in a portion of a medical device by contacting the portion with an agent that dissolves the portion to form the electrical discontinuity. For example, a stent can be implanted and expanded at a target site, and thereafter, an agent can be injected in to the target site to dissolve one or more regions of the stent. In some embodiments, the environment of a stent (e.g., after delivery to a target site) can cause one or more weak regions of the stent to dissolve. For example, one or more weak regions of a stent that has been delivered into the digestive tract may dissolve because of the relatively low pH of the region. In such embodiments, the weak region(s) of the stent may include a metal (e.g., magnesium, aluminum), and/or a polymer.

As an additional example, in certain embodiments, an electrical discontinuity can be formed in a portion of a medical device by heating the portion. For example, after the medical device has been delivered to a target site and expanded, the portion of the medical device can be heated (e.g., using an ablative laser) to melt the portion and form an electrical discontinuity. In some embodiments, the portion may have a lower melting point than other regions of the stent, such that, when exposed to heat, the portion begins to melt before the other regions of the stent.

As another example, in some embodiments, an electrical discontinuity can be formed in a portion of a medical device by cooling/freezing the portion. For example, after the medical device has been delivered to the target site and expanded, the portion of the medical device can be cooled/frozen. In some embodiments, the portion can be cooled/frozen using a cryo-balloon (a balloon that is filled with liquid nitrous oxide). The cooling/freezing of the portion can result in temperature-induced brittleness in the portion. Thus, the portion can break from strain caused, for example, by changes in artery shape due to heart beating and/or respiration. Examples of materials that can demonstrate brittleness at low temperatures include polymers, tantalum containing about 700 ppm oxygen or less, unrecrystallized molybdenum, and plain carbon and low alloy steels.

As a further example, in some embodiments, a weak region of a medical device can be formed out of a metal that has a relatively small grain size (e.g., less than about 45 microns), such that when the weak region breaks, it is relatively unlikely to form jagged and/or sharp edges. Examples of materials with relatively small grain sizes include tantalum, niobium, and titanium. In some embodiments, a material with a relatively small grain size can have a grain size of about 7.0 or more according to ASTM standard E112 (e.g., ASTM E112 G of from 7.0 to 9.0). In certain embodiments, the grain size of a material can be varied by, for example, localized treatment of the material. For example, localized heat treatment of a stent including a metal or metal alloy (e.g., stainless steel) can result in the metal stent having grain sizes ranging from 5.0 to 10.0 according to ASTM standard E112.

As an additional example, in certain embodiments, a weak region of a medical device can be formed out of a metal that has a relatively large grain size (e.g., ASTM E112 G of 1.0-3.0). In some cases, a region that is formed out of a metal with a relatively large grain size may be more likely to fracture at a lower strain value than a region with a relatively small grain size.

As another example, in some embodiments, one of the above-described stents can be a part of a stent-graft. In certain embodiments, a stent can include and/or be attached to a graft including a biocompatible, non-porous or semi-porous polymer matrix made of polytetrafluoroethylene (PTFE), expanded PTFE, polyethylene, urethane, or polypropylene.

As a further example, in some embodiments, a stent can include one or more releasable therapeutic agents, drugs, or pharmaceutically active compounds, such as anti-thrombogenic agents, antioxidants, anti-inflammatory agents, anesthetic agents, anti-coagulants, and antibiotics. For example, in embodiments in which the stent includes or more bioerodible portions, the bioerodible portions can include one or more therapeutic agents, drugs, or pharmaceutically active compounds. Therapeutic agents, drugs, and pharmaceutically active compounds are described, for example, in Phan et al., U.S. Pat. No. 5,674,242; Weber, U.S. Pat. No. 6,517,888; U.S. Patent Application Publication No. US 2003/0003220 A1, published on Jan. 2, 2003; and U.S. Patent Application Publication No. US 2003/0185895 A1, published on Oct. 2, 2003.

As an additional example, in some embodiments, one of the above-described stents can be coated. For example, the stent can be coated with a therapeutic agent, and can further be coated with a protective layer that is disposed over the therapeutic agent. Coated stents are described, for example, in U.S. Ser. No. 10/787,618, filed on Feb. 26, 2004, and entitled “Medical Devices”.

As a further example, in some embodiments, one of the above-described stents can be used in a magnetic resonance angiography (MRA) procedure. In such a procedure, the stent can be guided to an implantation site and implanted while being visualized with a magnetic-resonance scanner. In such embodiments, the stent can include one or more MRI-compatible materials, and/or can have a design that is conducive to visualization using magnetic-resonance imaging.

As an additional example, while stents including bioerodible metals and/or metal alloys have been described, in some embodiments, a medical device such as a stent can include other types of bioerodible materials. Examples of other types of bioerodible materials include polysaccharides (e.g., alginate); sugars (e.g., sucrose (C₁₂H₂₂O₁₁), dextrose (C₆H₁₂O₆), sorbose (C₆H₁₂O₆)); sugar derivatives (e.g., glucosamine (C₆H₁₃NO₅), sugar alcohols such as mannitol (C₆H₁₄O₆)); inorganic, ionic salts (e.g., sodium chloride (NaCl), potassium chloride (KCl), sodium carbonate (Na₂CO₃)); water soluble polymers (e.g., a polyvinyl alcohol, such as a polyvinyl alcohol that has not been cross-linked); biodegradable poly DL-lactide-poly ethylene glycol (PELA); hydrogels (e.g., polyacrylic acid, haluronic acid, gelatin, carboxymethyl cellulose); polyethylene glycol (PEG); chitosan; polyesters (e.g., a polycaprolactone); and poly(lactic-co-glycolic) acids (e.g., a poly(d-lactic-co-glycolic) acid). Bioerodible materials are described, for example, in U.S. Ser. No. 10/787,618, filed on Feb. 26, 2004, and entitled “Medical Devices”.

As another example, in certain embodiments, a weak region of a stent can be broken via electrolytic disintegration. When the stent is exposed to MRI, a current can flow through the stent, as described above. In some embodiments, as the current flows through the weak region of a stent, the current can cause the weak region to electrolytically disintegrate, thereby forming an electrical discontinuity that prevents the current from being able to travel in a continuous loop. Electrolytic disintegration is described, for example, in Guglielmi et al., U.S. Pat. No. 5,895,385.

As a further example, in some embodiments, one or more regions of a stent can be broken by exposing the stent to an ultrasonic frequency that corresponds to a natural harmonic frequency of the stent structure, resulting in stent fracture.

All publications, applications, references, and patents referred to in this application are herein incorporated by reference in their entirety.

Other embodiments are within the claims.

What is claimed is:

1. A medical device, comprising:
a member comprising a first portion and a second portion that define an electrically conductive loop,
wherein the first portion is adapted to break or erode after expansion of the medical device, and the second portion is not adapted to break or erode after expansion of the medical device, wherein the breaking or erosion of the first portion breaks the electrically conductive loop.

2. The medical device of claim 1, wherein the medical device does not define any electrically conductive loops after the first portion has broken or eroded.

3. The medical device of claim 1, wherein the first portion comprises a bioerodible material.

4. The medical device of claim 3, wherein the bioerodible material comprises a metal selected from the group consisting of magnesium, titanium, zirconium, niobium, tantalum, zinc, silicon, lithium, sodium, potassium, manganese, calcium, iron, and combinations thereof.

5. The medical device of claim 1, wherein the medical device is radiopaque.

6. The medical device of claim 1, further comprising an alloy comprising a metal selected from the group consisting of titanium, vanadium, tantalum, zirconium, niobium, molybdenum, platinum, palladium, aluminum, iridium, rhenium, tungsten, and combinations thereof.

7. The medical device of claim 1, further comprising an alloy selected from the group consisting of titanium-molybdenum, titanium-niobium-tantalum-zirconium, titanium-tantalum, titanium-aluminum-vanadium-tantalum, titanium-iridium, titanium-rhenium, titanium-tantalum-iridium, titanium-tantalum-rhenium, and niobium-zirconium.

8. The medical device of claim 1, further comprising a material having a magnetic susceptibility of less than about 0.9×10⁻³.

9. The medical device of claim 1, further comprising a material having a density of greater than about eight grams per cubic centimeter.

10. The medical device of claim 1, further comprising a metal or a metal alloy having a magnetic susceptibility of less than about 0.9×10⁻³ and a density of greater than about eight grams per cubic centimeter.

11. The medical device of claim 1, wherein the medical device comprises an implantable medical endoprosthesis.

12. The medical device of claim 11, wherein the implantable medical endoprosthesis comprises a stent.

13. The medical device of claim 11, wherein the implantable medical endoprosthesis comprises at least one band or strut defining a hole, a notch, a slot, a groove, or a chamfer.

14. The medical device of claim 11, wherein the implantable medical endoprosthesis comprises at least one band or strut having a first region with a first thickness and a second region with a second thickness that is greater than the first thickness.

15. The medical device of claim 1, further comprising an oxide.

16. A method, comprising:
expanding a medical device comprising a first portion and a second portion that define an electrically conductive loop,
wherein after the medical device has been expanded, the first portion breaks or erodes and thereby breaks the electrically conductive loop.

17. The method of claim 16, wherein first portion comprises a bioerodible material.

18. The method of claim 16, wherein the first portion comprises an oxide.

19. The method of claim 16, wherein the first portion has a first thickness and the second portion has a second thickness that is greater than the first thickness.

20. A method, comprising:
delivering a medical device into a lumen of a subject,
wherein the medical device comprises a member comprising a first portion and a second portion that define an electrically conductive loop, and the first portion is adapted to break or erode after expansion of the medical device, and the second portion is not adapted to break or erode after expansion of the medical device.

21. The method of claim 20, further comprising expanding the medical device.

22. The method of claim 21, wherein after the medical device has been expanded, the first portion breaks or erodes and thereby breaks the electrically conductive loop.

23. The method of claim 22, wherein the medical device does not define any electrically conductive loops after the first portion has broken or eroded.

24. The method of claim 22, wherein the electrically conductive loop is broken from about one week to about three weeks after the medical device has been expanded.

25. The method of claim 22, wherein the electrically conductive loop is broken from about one month to about three months after the medical device has been expanded.

26. The method of claim 22, wherein the electrically conductive loop is broken from about six months to about nine months after the medical device has been expanded.

27. The method of claim 21, further comprising, after the medical device has been expanded, expanding the medical device so that the electrically conductive loop breaks.

28. The method of claim 27, wherein the medical device is expanded using a medical balloon.

29. The method of claim 21, further comprising, after the medical device has been expanded, exposing the medical device to ultrasound.

30. The method of claim 21, further comprising altering a configuration of the medical device so that the electrically conductive loop breaks.

31. The method of claim 30, wherein altering a configuration of the medical device comprises breaking at least one component of the medical device.

32. The method of claim 31, wherein the at least one component comprises a band or a strut.

33. The method of claim 30, wherein altering a configuration of the medical device comprises heating a portion of the medical device.

34. The method of claim 30, wherein altering a configuration of the medical device comprises cooling a portion of the medical device.

35. The method of claim 30, wherein altering a configuration of the medical device comprises contacting a portion of the medical device with an agent that dissolves the portion of the medical device.

36. The method of claim 22, further comprising viewing the medical device with magnetic resonance imaging.

37. The method of claim 22, further comprising viewing the lumen of the subject with magnetic resonance imaging.

38. The method of claim 20, further comprising viewing the medical device using X-ray fluoroscopy.

39. A method, comprising:
expanding a medical device having at least one electrically conductive loop to break the at least one electrically conductive loop.
